(19) United States
(12) Patent Application Publication
(10) Pub. No.: US 2004/0009207 A1
(43) Pub. Date: Jan. 15, 2004

(54) Medicinal beverage containing molecular oxygen

(76) Inventors: Friedrich Schneider, Pullach (DE); Reinhardt Klimmek, Kruklanki (PL); Olaf Adam, Pocking (DE)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/380,155

(22) PCT Filed: Sep. 11, 2001
(86) PCT No.: PCT/EP01/10495

(30) Foreign Application Priority Data
Sep. 12, 2000 (DE) 100 45 022.9

(51) Int. Cl. A61K 47/00; A61K 33/00

(52) U.S. Cl. 424/439; 424/600

(57) ABSTRACT

The present invention relates to a medicinal beverage containing water and molecular oxygen physically dissolved therein for the use as a pharmaceutical product, wherein the amount of dissolved oxygen is at least 75 mg/l water and the uses thereof.

The present invention relates to a medicinal beverage containing water and molecular oxygen physically dissolved therein in a specific amount for the application as a pharmaceutical product.

In the past, water containing oxygen has mainly been used for treating and regenerating sewage. In U.S. Pat. No. 5,087,377, for instance, a device is described which allows for high-pressure oxygen saturation of the sewage that is to be treated. U.S. Pat. No. 5,525,242 discloses a method for the oxidative treatment of water contaminated with hydrogen sulphide gas or radon with a gas containing oxygen such as air.

While water containing oxygen is very common in the treatment of sewage, so far there has not been any scientific proof of a therapeutic effect of water enriched with oxygen and administered orally. Although in the general public opinion, oxygen is said to have a positive, generally advantageous effect on health, there is, however, no scientific proof of such an effect at all.

In a brochure called “water with naturally dissolved oxygen” (Dr. Ing. Princeton Christian Hechtl; 1999), which is distributed in beverage shops, “unimagined effects on the well-being, health and preservation of life” are mentioned, however without furnishing proof in detail by stating any data. 20 mg O₂/l water is stated as an upper limit that is still tolerable and, according to the author, can still be regarded as unrisky and that corresponds to the amount of oxygen which is dissolved in a natural mountain brook. In this brochure, it is expressively mentioned that higher dosages of up to 50 mg O₂/l water and more are to be avoided, since such dosages may allegedly lead to cancer.

U.S. Pat. No. 5,006,352 describes a method for producing beverages saturated with molecular oxygen as well as their use as strengthening and refreshing nutriment. Beverages produced according to said method contain between 32 and 35 mg O₂/l.

Furthermore, methods are known in which oxygen is administered as a gas.

In U.S. Pat. No. 4,027,045, for example, a method for producing beverages foamed with oxygen that are said to have a positive effect on the enteral oxygen supply is described. Although Ventricular ulcer, chronic gastritis and colitis, liver diseases, hypertension and arteriosclerosis are mentioned cursorily, there is no experimental or other proof of a medicinal effect whatsoever.

U.S. Pat. No. 5,086,620 discloses a method for encapsulating a hyperbaric gas for treating diseases in mammals with oxygen precursors, e.g. micro-capsules of oxygen, sodium superoxide, potassium superoxide or hydrogen peroxide.

To sum up, it can be said that, although food supplements enriched with oxygen are said to have a positive effect on health, a concrete medicinal effect proven by experimental facts has not yet been described. Since higher doses are not advisable due to the side effects to be expected, the oxygen content in common food supplements that are enriched with oxygen and that are on the market is between 5 and 60 mg O₂/l. A medicinal effect of food supplements having a higher oxygen content has not been known so far.

Moreover, gaseous oxygen, 21% (v/v) of which is contained in the atmosphere as a vital element, can have a toxic effect in higher doses or concentrations. Molecular oxygen is activated by means of mono-oxygenases, peroxidases and cytochromeoxidase-Fe³⁺.

These enzyme systems are present not only in the liver but also in many other tissues. Under certain circumstances, oxygen is harmful even in normal air, i.e. if the body tissues are deprived of their natural ability of reducing oxidation products, an example thereof being “spontaneous” methemoglobinemias formed due to a genetically caused lack of reductase. The strong concentration of CO₂ in tissues, in particular in the brain, is the reason for the toxicity of molecular oxygen, the reasons being: (1) Hb loses its capacity of binding CO₂ in venous blood, (2) the diffusion of CO₂ through the lung is impeded due to the formation of a typical toxic lung oedema. Such an effect detrimental to the lungs is of importance in the case of long-term exposure to pure or high-grade oxygen. If the lung is already damaged, even 60% O₂, over a several-hour period, is problematic. Thus, oxygen inhalations, with or without overpressure, e.g. in the case of poisonings, cardiovascular diseases, gas gangrene infections and lung affections should always be carried out with interruptions for the pulmonary alveoli structures to recover. The sensitivity as to O₂ varies strongly depending on the species. Although humans are not very sensitive, due to the possible negative consequences, methods based on gaseous oxygen bear risks which are to be avoided.

Many diseases result in a disorder in the oxygen supply of the inner organs. Depending on which of the organs is affected, this may have a negative effect on vital processes in the body. If the oxygen supply of the liver is impaired, essential metabolisms cannot not work at all or only at a lower pace. These diseases include swellings of the liver, e.g. due to the deposit of fat or the effects of toxic substances such as fibrous changes, the most extreme example thereof being liver cirrhosis. Due to the compression of the vessels, the blood circulation is impaired, or the continuous cicatricial connective tissue conversion of the liver leads to an increased constriction of the portal vein and, as a result, to portal hypertension and compression in the portal vein together with ascites due to transudation of the mesenteric veins (up to 15 l and more). As a consequence, the liver is not supplied with enough oxygen and many metabolic process which work oxidatively are impaired. Physiologically, there is, however, no way to improve the blood circulation of the inner organs, in particular of the liver, over a large circle. Neither is it possible to increase the blood circulation of the liver pharmacologically.

Therefore, the technical problem underlying the present invention is to provide a means for the prophylaxis and treatment of diseases or conditions connected with an unsatisfactory oxygen supply of the inner organs, wherein the means is not toxic, easy to be put into practice from a technical point of view and cheap.

This technical problem has been solved by the surprising finding that the oxygen partial pressure in the abdominal region and thus the oxygen supply of the inner organs, in particular or the liver, can be increased by means of the oral administration of water containing a specific amount of oxygen dissolved therein.

The liver is the central organ of metabolism of the body. There, nutrients are converted into utilisable forms or they are stored, and most of the toxic agents are made harmless. During the metabolism of carbohydrates, glycogen is formed which serves as a primary energy store and which provides phosphates rich in energy when being split up in a manner depending on oxygen. In a state of hunger, the liver uses the fats as its sole source of energy which it utilises by means of oxidation of fatty acids. During glycolysis and the oxidation of fatty acids, NADH and FADH₂, two molecules rich in energy, are formed which each have a pair of electrons with a great transfer potential. During the transfer of these electrons to molecular oxygen, a large amount of energy is released which may be used for producing ATP. This process is called oxidative phosphorylation. It supplies ATP to an extent to which electrons are transferred from NADH and FADH₂ to oxygen in the mitochondrial respiratory chain. In the liver, too, phosphates rich in energy are required for the synthesis of proteins and of a vast amount of molecules. In addition, the liver serves for the detoxification of pharmaceutical compositions and pollutants by coupling with hydrophilic residues resulting in a transfer of the substances to water-soluble substances. In the liver, natural and synthetic pharmaceutical compositions and toxins are mostly metabolised first in a hydrolytic, oxidative or reductive manner and are then esterified with glucuronic acid, sulphate, acetate, glycine or methyl groups for better elimination. These so-called conjugation reactions also require ATP. The enormous performance of the liver as regards metabolism requires a constant supply with oxygen.

Oxygen is transported to the liver via the blood. The liver contains blood from two sources:

oxygen-deficient (venous) blood from the portal vein (Vasa publica) from which about 75% of the liver blood volume are derived and with which half of the oxygen demand is covered, and

blood rich in oxygen (arterial blood) from the liver artery (Vasa privata) from which about 25% of the liver blood volume are derived, with which, however, half of the oxygen demand is covered.

The arterial supply of the liver takes place via the Arteria hepatica which has its source in the Truncus coelacus or the superior mesenteric artery (Arteria mesenterica superior). The body does not have any possibility of enhancing the oxygen supply of the liver by increasing the blood flow in the Arteria hepatica beyond the normal range. If an increased amount of oxygen is needed or if the blood supply via the Arteria hepatica is restricted, the liver is dependent on an increased oxygen supply from the portal vein. The velocity of flow in the portal vein, however, is so low that the oxygen supply can only be increased to an insignificantly low extent in this manner.

Virtually all the venous blood of the stomach flows via the four large gastric veins to the portal vein. The veins of the duodenum partly flow into the Vena mesenterica superior, partly directly into the portal vein. The superior mesenteric veins unify with the splenic vein to form the portal vein. In the liver, the portal vein and the arterial branches are always close to each other. While in the rest of the body, veins form bigger and bigger trunks and lead the blood back to the heart, the portal vein, which resulted from the intestinal veins, splits into a capillary network in the liver from which the liver veins originate (the so-called “miraculous network” of the liver).

The oxygen is transported in the organism by binding to haemoglobin. In the organism, however, the haemoglobin does not only serve the purpose of transporting oxygen, but also of transporting CO₂ in the blood. When O₂ is bound, haemoglobin goes through a conformation change under physiological pH values, by which the acidity is increased and protons are released.

Hb(O₂)ₙHₓ+O₂ [symbol] Hb(O₂)ₙ₊₁+xH⁺

wherein n=0, 1, 2, 3 and x=0.6.

If the pH value is increased, e.g. by trapping protons, the capacity of binding O₂ of the haemoglobin is increased (Bohr effect). Per 1 M O₂, 0.8 M CO₂ are formed which diffuse from the tissue into the capillaries preferably in a dissolved form, since the formation of hydrogen carbonate is very slow:

CO₂+H₂O [symbol] H⁺+HCO₃—.

In the blood, this reaction is catalysed by carbonic anhydrase. The majority of CO₂ is transported as hydrogen carbonate in the blood. In the capillaries, the oxygen partial pressure is low; Hb is protonated by the protons released by the formation of hydrogen carbonate and increasingly releases O₂. The formation of hydrogen carbonate is promoted due to the consumption of H⁺. In the lung, however, the oxygen partial pressure is high; Hb is loaded with O₂ and, due to the Bohr effect, protons are released. Carbonic acid is formed which decomposes into water and CO₂ which is then exhaled.

Therefore, a medicinal beverage containing water and molecular oxygen physically dissolved therein for use as a pharmaceutical product is the subject matter of the present invention. Preferably, the amount of oxygen dissolved should be larger than 75 mg/l, in particular more than 80 mg/l, more preferably 85 to 600 mg/l and most preferably 85 to 400 mg/l.

Surprisingly, an unexpected increase of the oxygen partial pressure in the abdominal region can be observed when the medicinal beverage is administered.

While even low doses of dissolved oxygen can lead to an increase in the oxygen content in the venous blood, the clinical relevance of such an increase is comparatively small since, in particular in vessels with a low velocity of flow, it is not possible to sufficiently increase the oxygen supply. Moreover, the oxygen supply in the smallest venous vessels, the so-called venules, cannot be increased in this manner since, during transport, the oxygen content decreases and, thus, is not constant long enough for all cells to be reached via the capillaries. If, however, amounts according to the invention of dissolved oxygen is administered orally, the oxygen partial pressure in the abdominal region is unexpectedly increased in a manner that is of clinical significance.

Due to the high oxygen partial pressure in the lung, on the one hand, and the external surroundings on the other hand, diaphragm and the abdominal wall are an insurmountable membrane for the oxygen, so that the oxygen partial pressure in the abdominal region remains high for a long time and may be distributed into the vessels of the abdominal region, including the venules, as well as into the inner organs, in particular into the liver.

Although the increase in the oxygen partial pressure in the abdominal region depends on the dose, the effect on oxygen concentrations below 75 mg/l is insignificantly small. In an animal experiment, it could be seen that, if a beverage having a oxygen content of 45 mg/l was administered, the partial pressure in the abdominal region is insignificantly low, whereas, if a beverage of the invention having an oxygen content of 150 mg/l was administered, the partial pressure could be increased to 58 mmHg.

As a consequence, the preferred amount of dissolved oxygen in the beverage of the invention is at least 75 mg/l, more preferably at least 80 mg/l and most preferably 85 to 600 mg/l water.

Surprisingly and contrary to the common opinion, the oral administration of dissolved oxygen does not have any detectable side effects even if very high doses are administered.

Furthermore, according to the invention, it was found that the oxygen partial pressure in the abdominal region, and thus the oxygen supply of the vessels of the abdominal region and the inner organs, can be increased further if carbon dioxide is added to the beverage containing oxygen.

Thus, in addition, a medicinal beverage containing water and molecular oxygen physically dissolved therein and carbon dioxide physically dissolved therein is also a subject matter of the invention. The amount of dissolved oxygen is at least 75 mg/l, preferably at least 80 mg/l, more preferably 85 to 600 mg/l and most preferably 90 to 400 mg/l. The amount of dissolved carbon dioxide may be up to 8000 mg/l. Preferably, the amount of carbon dioxide is up to 3000 mg/l, more preferably 1000 to 2000 mg/l.

Furthermore, the use of the medicinal beverage (with or without carbon dioxide) as a pharmaceutical product for the treatment and prophylaxis of conditions and diseases which accompany a lack of oxygen supply of the inner organs as well as for increasing the oxygen partial pressure in the abdominal region, including the inner organs, is also a subject matter of the invention.

Surprisingly, it was found that the oxygen supply of the inner organs, in particular of the liver, may be increased by the administration of the medicinal beverage of the invention which contains water and molecular oxygen physically dissolved therein in an amount of at least 75 mg/l, preferably at least 80 mg/l, most preferably more than 85 mg/l. Without limiting to one specific theory, it is to be assumed that, in the case of oral administration of the medicinal beverage, there is a strong increase in the gastrointestinal oxygen pressure induced by the rise in temperature during uptake, so that a sufficient amount of oxygen can diffuse through the gastric mucosa. In this way, the oxygen partial pressure in the abdominal region unexpectedly rises slowly and continuously. Due to the high oxygen partial pressure in the lung on the one hand and the external surroundings on the other hand, the diaphragm and the abdominal wall are an insurmountable membrane for the oxygen, so that the oxygen partial pressure in the abdominal region remains high for a long time and is redistributed into the vessels and the very small vessels and the inner organs as well as into the inner organs, in particular into the liver. If the oxygen supply via the lung is interrupted or if the patient suffers from hypoxia, the O₂ from the abdominal region is also utilised via the lung. The diaphragm is then permeable for O₂.

In principle, there are no restrictions as to the water used for the present invention as long as it is well-tolerated as to health. Tap water, spring water, mineral water or other kinds of medicinal water may, for instance, be used as a basis for the medicinal beverage of the present invention. The water may be degased or may be used in its natural state.

In addition, it is not important in which manner the oxygen is dissolved in the water as long as the amount required can be dissolved. The oxygen may be added, for instance, in form of pure oxygen gas or in form of gas mixtures, e.g. air or other mixtures of, for example, nitrogen and oxygen. In the present invention, the oxygen is preferred to be added as a pure oxygen gas, however, provided that the gas used is well-tolerated from point of view of health.

In the present invention, the amount of dissolved oxygen is at least 75 mg/l, preferably at least 80 mg/l, more preferably 85 to 600 mg/l water. The advantageous effects of water enriched with molecular oxygen administered orally can only be seen if the amount of oxygen exceeds 75 mg/l, preferably 80 mg/l. The resistance of the tissue diffusion of molecular oxygen through the gastric mucosa is significantly higher than the one of carbon dioxide (30 times as high). Therefore, if the amount of dissolved oxygen is less than 75 mg/l water, no sufficiently high increase of the oxygen partial pressure can be induced in the stomach in the case of oral administration, so that the oxygen is only resorbed in the venous vessels of the abdominal wall, however, it is not possible to achieve diffusion through the gastric mucosa and to increase the oxygen partial pressure in the abdominal region at all or only to an insignificantly low extent. Although in the case of amounts of dissolved oxygen of less than 75 mg/l, an increase in the oxygen content can be measured in the venous blood, said increase is only very low and clinically not relevant. Furthermore, the velocity of flow in the venous blood vessels supplying the liver is so low that it is not possible to sufficiently increase the oxygen supply in this way. This is why it is essential that the amount of enriched oxygen is high enough for the tissue resistance of the gastric mucosa to be overcome and for the unexpected increase in the oxygen partial pressure in the abdominal region to be induced. Only then is it possible to sufficiently increase the oxygen supply of the vessels and smallest vessels as well as the inner organs, in particular the liver, in order to achieve the positive medical effects.

The amount of dissolved oxygen preferably is 85 mg/l to 400 mg/l, in particular 90 to 300 mg/l, more preferably 90 to 200 mg/l and most preferably 100 to 200 mg/l.

Moreover, the invention relates to a medicinal beverage containing water, oxygen physically dissolved therein and carbon dioxide physically dissolved therein. In this case, the amount of dissolved oxygen is, according to the invention, at least 75 mg/l, preferably at least 80 mg/l, more preferably at least 85 mg/l, particularly 85 to 600 mg/l, still more preferably 85 to 400 mg/l, particularly 90 to 300 mg/l and most preferably 90 to 200 mg/l. The amount of dissolved carbon dioxide may, for instance, be up to 8000 mg/l. Preferably, the amount of dissolved oxygen is up to 3000 mg, more preferably 500 to 3000 mg, most preferably 1000 to 2000 mg per litre water. In a preferred embodiment, the content of oxygen is 90 to 200 mg/l and the content of carbon dioxide is 1000 to 2000 mg/l.

Surprisingly, it was found that the oxygen partial pressure in the abdominal region can still be increased if carbon dioxide is added to the beverage containing oxygen. In this case, the amount of oxygen administered orally can be lower than is the case if dissolved oxygen only is administered, however, it is at least 75 mg/l. The oral administration of water in which at least 75 mg O₂/l and, at the same time, carbon dioxide are dissolved has a synergistic effect. If dissolved oxygen and dissolved carbon dioxide are administered simultaneously, the increase induced in the abdominal region surprisingly is higher than is the case if dissolved oxygen only is administered.

The effect observed can possibly be explained by the fact that, if the pH is low and the carbon dioxide partial pressure is higher, the oxygen-binding curve of the haemoglobin is shifted to the right compared to arterial blood, i.e. the binding of oxygen to haemoglobin has become more difficult. Due to the carbon dioxide partial pressure in the mesenteric veins, which is higher than in the arteries, the increase of oxygen binding in the mesenteric veins should also be impaired. If oxygen is administered at the same time, the O₂ reservoir of the blood can be refilled by way of diffusion via the gastric mucosa in the abdominal region and, from there, to the surrounding tissues and vessels.

Apart from oxygen and water, the medicinal beverage (with or without carbon dioxide) may also contain minerals such as sodium ions, potassium ions, magnesium ions, calcium ions, strontium ions, fluoride ions, chloride ions, nitrate ions, sulphate ions, carbonate ions, hydrogen carbonate ions, metasilicate, metaboric acid and combinations thereof.

Moreover, it is possible to add food supplements, e.g. vitamins, amino acids, sweeteners and trace elements as well as flavouring substances. The amount of additives should be selected in such a way that the necessary amount of oxygen can be dissolved in the solution, preferably the amount of additives is selected in such a way that the preferred amount of oxygen and the preferred amount of carbon dioxide can still be dissolved.

In addition, the medicinal beverage may contain further medicinal active agents such as anti-inflammatory agents or anti-tumour agents.

The medicinal beverage of the invention (with or without dissolved carbon dioxide) may be used as a pharmaceutical product itself or as a vehicle for further pharmaceutical products for the treatment and prophylaxis of diseases and conditions accompanying the insufficient oxygen supply of the inner organs.

Within the meaning of the invention, the term “conditions” are such states that cannot yet be designated pathogenic and that accompany an insufficient oxygen supply of the inner organs. Such conditions may, for example, be metabolic complications as a consequence of poisoning or of an overdose of a medicament. In particular, such conditions are expected to occur if pharmaceutical products, drugs or poisons or other substances are administered which utilise oxygen during metabolisation of detoxification, i.e. they are metabolised oxidatively.

Examples thereof include substances the metabolisation of which is catalysed by mixed-functional oxygenases, e.g. by cytochrome P-450. In this case, an oxygen atom from molecular oxygen is transferred from cytochrome P-450 to the substrate. The other oxygen atom is reduced to water. Typical substrates of these mixed-functional oxygenases are, for instance, aromatic hydrocarbons, heterocyclic amines, aflatoxin B₁, caffeine, coumarin, diethyinitrosamine, cyclophosphamide, tolbutamide, Rmephenytoin, warfarin, debrisoquin, sparteine, ethanol, acetone, benzole, chloroform, nitrosamine, chloroxazon, dihydropyridine, cyclosporin, triazolam.

Flavin-containing mono-oxygenases take effect by oxidation. These microsomal enzymes, e.g. FAD-flavoprotein, compete with cytochrome P-450, in particular as regards the oxidation of amines. These include, amongst others, tertiary amines, e.g. chlorpromazine, morphine or nicotine and secondary amines, e.g. propanolol, desipramine and metamphetamine.

The oxidation by peroxidases also depends on oxygen and relates to substances and pharmaceutical compositions which serve as co-substrates during oxidation. These may, for instance, include polycyclic aromatic hydrocarbons, aromatic amines, nitrofuranes, non-steroidal anti-inflammatories, diethylstilbestrol and aromatic sulphides.

An insufficient oxygen supply also occurs in the case of poisonings, e.g. with carbon monoxide or cyanides. Also in the case of excessive alcohol consumption, there is an insufficient oxygen supply in the liver due to oxidative metabolisation of ethanol to acetic acid.

Thus, the beverage of the invention is particularly suitable for the treatment and prophylaxis of liver damage connected with alcohol.

Another use of the medicinal beverage of the present invention is the use of the medicinal beverage as a pharmaceutical product or for producing a pharmaceutical products for increasing the oxygen partial pressure in the abdominal region, in the inner organs located therein and in the vessels.

Inner organs are, in particular, the liver, the spleen, the kidney, the stomach, the intestines, the pancreas and the gallbladder. The administration of the medicinal beverage has a particularly positive effect on the oxygen supply of the liver and the metabolic processes taking place there.

The administration of the medicinal beverage in which oxygen and carbon dioxide are dissolved in amounts according to the invention, also has a particularly positive effect on the oxygen partial pressure of the vessels of the abdominal region. These are known to the person skilled in the art and include, e.g. the gastric veins, e.g. the Vena gastica sinistra, the Vena gastrica dextra, the Vena gastro-omentalis [gastro-epiploica] sinistra and the Vena gastro-omentalis [gastro-epiploica] dextra, the liver veins, e.g. the Venae hepaticae dextrae, intermediae and sinistrae, the Vena portae or the capillaries and venules of the mesentery and the peritoneum.

So far no possibilities of significantly increasing the oxygen partial pressure in these vessels has been known. By administering the medicinal beverage, the oxygen partial pressure in the abdominal region is increased and is redistributed from there to the vessels and organs of the abdominal region. The increase in the oxygen partial pressure in these vessels has a positive effect in the organs supplied by these vessels.

In cases in which the supplying vessels are pathologically changed due to diseases, e.g. if they are narrowed or blocked, so that the supplied amount of blood and thus also the supplied amount of oxygen is reduced, particularly positive results have been achieved.

Such changes are known mainly in connection with diseases which are accompanied by a reduced supply of oxygen in the blood or with a reduction of the blood supply of the liver, which, as a consequence, reduces the amount of oxygen available for the liver. This may also be the case if the velocity of flow in the portal vein is too low due to an increased pressure in the hepatic circulation.

Such changes are known, for example, with diseases such as arteriosclerosis, all kinds of portal vein hypertension, e.g. hepatic fibrosis, hepatic cirrhosis, fatty liver, toxic hepatitis, infectious hepatitis and storage diseases, ischemic diseases of the intestine and their consequences as well as all diseases and conditions which accompany an increased need for energy of the liver, e.g. malnutrition, inflammations, intoxications, and with drug therapy.

A preferred use of the medicinal beverage of the present invention relates to the use for treating and preventing hepatic diseases. Hepatic diseases are known to the person skilled in the art and comprise, for instance, hepatic cirrhosis, hepatic abscess, hepatic infarction, hepatic necrosis, fatty degeneration of the liver, hepatic tumours, hepatitis, hepatic coma and hepatic insufficiency.

Hepatic cirrhosis may include any form of cirrhosis, e.g. alcoholic cirrhosis, post-hepatic liver cirrhosis, cryptogenic hepatic cirrhosis, metabolic hepatic cirrhosis, hepatic cirrhosis with hepatolentricular degeneration, hepatic cirrhosis with glycogenosis, hepatic cirrhosis with galactosemia, hepatic cirrhosis with Debré-Toni-Franconi syndrome, hepatic cirrhosis with cystic fibrosis, cardiovascularly caused hepatic cirrhosis, cardial hepatic cirrhosis with right-ventricular heart failure and panzerheart as a final state of chronic stasis liver, hepatic cirrhosis with Budd-Chiari syndrome, hepatic cirrhosis with Osler-Rendu-Weber disease or hepatic cirrhosis as a consequence of chronic hepatitis.

A hepatic abscess may be both a pyogenic hepatic abscess and an amebic abscess.

A hepatic infarction may be a haemorrhagic hepatic infarction with blockage of a branch of the portal vein, an anaemic hepatic infarction with blockage of a branch of the Arteria Hepatica, and a fatty infarction.

Hepatic necroses occur with the necrotising form of acute hepatitis or with grave hepatoses, e.g. with mushroom poisoning with Amanita Phalloides.

Fatty liver occurs, for example, with diabetes mellitus or increased alcohol consumption.

A preferred use of the medicinal beverage of the invention relates to the prophylaxis of neoplastic processes. The increase in oxygen supply thanks to the medicinal beverage reduces the anaerobic metabolic processes in the degenerated cells and can, thus, contribute its share to the reduction of the tumour growth and the prevention from metastasis. Therefore, a better supply with oxygen does not only have a preventive effect as regards tumour formation, but it can also contribute its share to the prevention of growth and metastasis of neoplasias in the abdominal region.

Examples of tumours the growth and metastasis of which can be reduced or prevented by administration of the medicinal beverage include, for instance, neoplasias of the liver. These kinds of cancer mainly develop in the liver degenerated due to a cirrhosis or in a liver that is already damaged. Further tumours of the abdominal region which may be influenced by an increased supply with oxygen include neoplasias of the bile duct system, neoplasias of the gastrointestinal tract, peritoneal carcinomas, ovarian carcinomas, uterus neoplasias and neoplasias of the ureteral ducts. It could be shown in an experimental approach that the sensibilisation of the tumour to radiation therapy or chemotherapy is increased if oxygen is administered at the same time. Without oxygen, for instance, a threefold radiation dose is necessary for killing the same number of tumour cells.

In a preferred embodiment, the medicinal beverage is used for preventing or reducing the tumour growth and for preventing metastasis, in particular of tumours for which P53 and VEGE are of importance for the malign progression. P53 is of importance mainly as regards the frequent kinds of intestinal cancer, the colon carcinoma and the ovarian carcinoma.

When the medicinal beverage of the invention is administered, in particular in the treatment and prophylaxis of metabolic diseases or conditions with disturbed metabolism, positive effect have been achieved. Such diseases and conditions include, for instance, diabetes mellitus, arteriosclerosis, congenital metabolic disorders with reduced glucose utilisation, disorders of the lipolysis, hyperlipidemia, conditions with increased need for energy, e.g. all fibrous conditions, burns, catabolism, stress, therapy with catabolic pharmaceutical products such as cortisone or anti-tumour agents, as well as conditions with exogenically increased metabolism, e.g. huge work load, high-performance sports or pregnancy.

Finally, the enrichment with molecular oxygen in the abdominal region leads to an oxygenation of the venous flow and, thus, to an improved supply with oxygen in the entire organism. This may have a particularly positive effect if the supply with oxygen by the lung is reduced, e.g. in the case of asthma bronchiale or obstructive lung diseases, wherein, in patients affected, an improved oxygen supply is reckoned to result in a reduction of pharmaceutical products. In the case of peripheral blood circulation disorders, the administration of the medicinal beverage may have a positive effect on the improvement of the flow as well as on the improvement of ulcera in connection with the blood circulation. Finally, the increased oxygen supply in the liver leads to an improved utilisation of fat and glucose resulting in a generally increased capacity.

A particularly preferred use of the medicinal beverage of the present invention relates to the use for preventing diseases and conditions which accompany an insufficient oxygen supply of the inner organs and vessels or an increased need for oxygen.

In another preferred use, the medicinal beverage is used for increasing the performance. Although conventional oxygen-containing food supplements are said to have a general performance-boosting and stimulating effect, this effect is much smaller than the effect that may be achieved with the medicinal beverage of the present invention by increasing the oxygen partial pressure in the abdominal region by which all cells can be supplied with oxygen via the capillaries.

Production of a Medicinal Beverage

The solubility of gases in water can be determined according to the Dalton-Henry law. According to this law, the solubility of a gas at a given temperature is proportional to its pressure. Thus, in one litre water, 49.1 ml O₂ dissolve at 0° C., at 50° C., however, only 20.9 ml dissolve, independently of the pressure.

According to this law, it is preferred to carry out the production of the medicinal beverage under increased pressure and at a low temperature. If this parameter is varied, the amount of oxygen to be dissolved can be adjusted to the desired value.

In a preferred embodiment, the water temperature during the enrichment with oxygen is less than 18° C., preferably less than 16° C., more preferably 0.5-10° C.

The pressure to be applied can be selected at will. In practice, however, there may be restrictions due to the devices used. The pressure can, for instance, be 0.5 to 15 bar. In a preferred embodiment, the pressure is 1 to 7 bar.

If the beverage is, at the same time, enriched with carbon dioxide, the order of enrichment may be selected at will. Firstly, for example, the enrichment with the desired amount of carbon hydroxide may be carried out and then the desired amount of oxygen may be dissolved. It is also possible to change this order. Both gases can also be added simultaneously, either individually or as a defined mixture.

In a preferred embodiment, CO₂ and O₂ are mixed in a defined mixture ratio and the gas mixture is added to the water (impregnated) at a temperature of 5 to 18° C. and under a pressure of approx. 1 to 4 bar. A suitable mixture ratio is, for instance, 98 to 80% (v/v) CO₂ and 2 to 20% (v/v) O₂.

For balancing variations in pressure during the enrichment process, it may be appropriate to use an intermediary container in the enrichment device for buffering.

The medicinal beverage may be filled into any container, e.g. glass or plastic. If large amounts of oxygen are to be dissolved, containers made of oxygen-impermeable plastic material are preferred to be used, e.g. combined material of PET and nylon (e.g. PET-nylon-PET). The size of the packing can be selected at will and can, for instance be 0.5 l to 1.0 l.

The invention is explained in more detail by way of the following examples and experiments.

### EXAMPLES

1. Production of a Medicinal Beverage According to the Invention Containing O₂.

Degased water (4.6 mg sodium ions, 0.5 potassium ions, 31.0 mg magnesium ions, 94.0 mg calcium ions, 0.17 mg strontium ions, 0.8 mg barium ions, 0.08 mg fluoride, 3.6 mg chloride, 4.0 mg nitrate, 10.0 sulphate, 9.4 mg metasilicate and 0.12 mg metaboric acid) is placed in a tank. The water is cooled to a temperature of 8° C. Under a pressure of 3.5 to 4 bar (initial pressure 5 bar) pure oxygen is enriched. The enriched water is buffered in a storage container and filled. The water contains 200 mg O₂.

2. Production of a Medicinal Beverage of the Invention Containing O₂ and CO₂.

Degased water (4.6 mg sodium ions, 0.5 potassium ions, 31.0 mg magnesium ions, 94.0 mg calcium ions, 0.17 mg strontium ions, 0.8 mg barium ions, 0.08 mg fluoride, 3.6 mg chloride, 4.0 mg nitrate, 10.0 sulphate, 9.4 mg metasilicate and 0.12 mg metaboric acid) is placed in a tank. The water is cooled to a temperature of 9° C. Under an initial pressure of 12 bar, CO₂ and O₂ are mixed in a defined manner (96.5% CO₂ and 3.5% O₂). Under a pressure of 1 bar (initial pressure of 4 bar), the water is impregnated with the gas mixture. The enriched water is buffered in a storage container and filled. The water contains 1.8 g CO₂ and 100 mg O₂.

3. Determination of the Oxygen Adsorption of Water Enriched with Oxygen.

30 ml water enriched with oxygen are applied to an anaesthetised rabbit lying on the right side directly into the stomach via a pharyngeal probe. The oxygen partial pressure and the carbon dioxide partial pressure as well as the pH value in the abdominal region were measure. For this purpose, a Paratrend probe was placed next to the stomach in the epigastrium via a line that was laid during surgery. In an interval of 15 seconds and over a period of approx. one hour, the parameters pH, pCO₂ and pO₂ were measured via a probe and recorded. The oral administration of the medicinal beverage of the invention leads to a continuous increase in the oxygen partial pressure from 31 to 58 mmHg in the abdominal region. At the end of the experiment (after one hour) a plateau formation of the pO₂ became apparent: the value achieved after this period of time remained the same during the further test period.

During the experiment, a slight decrease of the carbon dioxide partial pressure from 61 to 58 mmHg could be observed. During the entire experiment, the pH remained the same so that the changes found for pCO₂ and pO₂ can be attributed exclusively to the amount of oxygen administered orally and dissolved in the water.

4. Utilisation of the Oxygen from the Abdominal Region.

30 ml water with 150 mg/O₂ and 1.3 g/l CO₂ were administered to an anaesthetised rabbit lying on the right side into the stomach via a pharyngeal probe. For recording pH, pCO₂ and pO₂, a Paratrend probe was placed next to the stomach in the epigastrium via a line that was laid during surgery. In an interval of 15 seconds and over a period of approx. one hour, the parameters pH, pCO₂ and pO₂ were measured via a probe and recorded. As in experiment 3, a significant increase of the oxygen partial pressure in the abdominal region could be observed. The rabbit was monitored over a period of about 2 hours after the beginning of the measurement. After about 1.5 hours, the rabbit woke up from the anaesthesia and started to move. A sharp fall of the pO₂ in the abdominal region to the starting value is an indication of the utilisation of the oxygen from the abdominal region.

The experiments show that the oral administration of the beverage of the invention leads to an increase in the oxygen partial pressure in the abdominal region. This increase is steady. After reaching a plateau value, the oxygen partial pressure remains high over a longer period. If energy is consumed, e.g. as a result of movement, the oxygen stored in the abdominal region is quickly utilised. After a short while, the oxygen concentration falls to the starting value.

1. A medicinal beverage containing water and molecular oxygen physically dissolved therein for the use as a pharmaceutical product, wherein the amount of dissolved oxygen is at least 75 mg/l water.

2. The medicinal beverage according to claim 1 further containing carbon dioxide.

3. The medicinal beverage according to claim 1 or 2, wherein the amount if dissolved oxygen is 75 to 600 mg/l water.

4. The medicinal beverage according to claim 2 or 3, wherein the amount of oxygen dissolved in carbon dioxide is up to 8000 mg.

5. The medicinal beverage according to claim 2 or 3, wherein the amount of dissolved carbon dioxide is 500 to 3000 mg.

6. The medicinal beverage according to any one of claims 1 to 5 furthermore containing one or more substances selected from minerals, trace elements, vitamins, amino acids, flavouring agents and sweeteners and combinations thereof.

7. Use of a medicinal beverage containing water and molecular oxygen physically dissolved therein for treating and preventing diseases and conditions which accompany an insufficient oxygen supply of the inner organs.

8. Use of a medicinal beverage containing water and molecular oxygen physically dissolved therein for increasing the oxygen partial pressure in the abdominal region, in the inner organs and in the vessels located in the abdominal region.

9. Use according to claim 7 or 8, wherein the inner organs are liver, spleen, kidney, stomach, intestine and gall.

10. Use according to claim 7, wherein the diseases are hepatic diseases.

11. Use according to claim 10, wherein the hepatic diseases comprise hepatic cirrhosis, hepatic abscess, hepatic infarction, hepatic necrosis, fatty degeneration of the liver, hepatic tumours, hepatitis, hepatic coma and hepatic insufficiency.

12. Use according to claim 7 for the prophylaxis of liver damage caused by alcohol.

13. Use according to claim 7 for preventing the formation of tumours and for reducing the growth of metastases of neoplasias in the abdominal region.

14. Use according to any one of claims 7 to 13, wherein the beverage additionally contains dissolved carbon dioxide.
